SEQUENCE LISTING (5 sequences)

SEQ ID NO: 1 - DNA, 4724 bp, Brassica napus
  misc_feature 10, 15, 30: n = A,C,T or G
tctctagatncgganccattttctcagatnctcttatctttttcttcttgtttctaggttatcttgaagccactttctcctctttgtctctataagcaaagcaatagcatacataacacaatcaatcactcatcttcctataccttattacaaaactaaagcatgaagcttcctcgtaacatagacttcactaacattgcttactgcagttgtaacgtgagtattgtccgcctgagatccttctcttgtaagcctatggtcccatattttctgaaaaataattagttaacagcacgttagaagatttgaatatcaccttcccaaccaaatatattacacactatcatccattgaaccacatattaaccaaacattggctgttagagaatccagcacacaaatttctccccaagatatcgcaatacagttcacagaagtcagactcctagtctaatgttcgtgggccatagtcgaacatatatatacagtaagtgtgttaagttgttccttcattaactttcaagaactgtcatgatcagaatcgaccaagtttcctataaagtggtttttaggttttaagattttaccttaacttaatgtaaassagacaatacttcagtggcaccaagcatgatccaaaggctttgtttccatggtaacgtgcaagctcaccgaaggggtctcactgaagaagctgtacctcgtcctcgtcttcttttccggcctctaactgaaatggaatatccagtggtcgcttatatatggttgaaaaacaaatgagtgaattatctattccagcacaaagcaagtatttgaaccatatggtcaacgtgcgtaatgttcttacgttggaagatcttgcttggttagtttgggataagtaacaatgcagcatcaaagtctccttgacaatctaaggctttccatatagttcaagacaccagagaagctgggaacttttcttggaggtcagacatattgctcgccaaagctttgtggagtaccccatcatcagaaagcgcaatccaagcaccaaaatcatcagtagcactcgccgttcttcttgaagctgatttactttcacaccatttgtggaaatatgtttcctttaagagtcagatggtccagctaagaaaagggtaagctcattacgatcatcttgatcatcttcgtccaaacgaacccgtctcggagctcctttgctagcttctttgtgagacttctctatcttcacaacctgagtctccatagcgaaagtaaactcacgatgcctgtctcgagtttcgtctttcaaaccattgtcgtagctagattgaacatcattttggaactcatctaagtacagagaaacgatactggcaatcaataaacgttatacaataagacaatatgagagcagagaaggagataaagaatgaaacctgccatttggtggttccagagagcgtcgtgaaggtccctgtggagctgatcttaccgaccgaacctcttcgccatctcctgagatttttagaaaatggaaagaagggaagcaaaagagaataatcatggatttgaataactggaaacaataaagtaataaaagattcaaggaatcgcttaatcagaaatcaaagtgtaaaaaaaaattagaaaccataaagtaataataggaaatctccaattaatttgccaagacatatcacctgcgatgtattcatacacattccgagtgatagagaaggaaccgtttccgttgatagaagagaaggcaaaacgtcaagattgatgatctacaaaggaaatcatcacgtgaattgaaatctcgaagttccatagctggttacagagatcgaaggagaagccttttcgattcagtgtggaagaagaacaggaactctaaagtttgtgctttggtcttgctggccaaactcagagcccaacgtaataaccaacggaagcccatcaacatttgcgtttaatgaaaccatgagtttcgttcctgaatgacatgtgtcagcacgggggaaatccacttatctatataaagtttcacgctcaagttgaacgacttagtaacatggaagctgaggtagatagtctaagagtgaagaaaattcttctttataataatagatattgtagcctaaacgttcttagtgcacatttttaaatgcaaatatattacggatttggtcctaatttctgtaaaataaaactacttcattgttttatataaataactctattgctgcagtttcccaactttgttgcttagattcaggtctcacaatcaagaaatcaagaacagttagatccacaaattctacattttatttaataagtgaagtgacaaaaaacgagattagattcgtttctattcatccataattaataaaaaaaaaagaccaaacaaacaaaaatcatattccaagggtattttcgtaaacaaacaaaaccctcacaagtctcgttttataaaacgattcacgttcacaaactcattcatcatctctctctcctctaaccatggcggcggcaacatcgtcttctccgatctccttaaccgctaaaccttcttccaaatcccctctacccatttccagattctcccttcccttctccttaaccccacagaaagactcctcccgtctccaccgtcctctcgccatctccgccgttctcaactcacccgtcaatgtcgcacctccttcccctgaaaaaaccgacaagaacaagactttcgtctcccgctacgctcccgacgagccccgcaagggtgctgatatcctcgtcgaagccctcgagcgtcaaggcgtcgaaaccgtctttgcttatcccggaggtgcttccatggagatccaccaagccttgactcgctcctccaccatccgtaacgtccttccccgtcacgaacaaggaggagtcttcgccgccgagggttacgctcgttcctccggcaaaccgggaatctgcatagccacttcgggtcccggagctaccaacctcgtcagcgggttagcagacgcgatgcttgacagtgttcctcttgtcgccattacaggacaggtccctcgccggatgatcggtactgacgccttccaagagacaccaatcgttgaggtaacgaggtctattacgaaacataactatttggtgatggatgttgatgacatacctaggatcgttcaagaagctttctttctagctacttccggtagacccggaccggttttggttgatgttcctaaggatattcagcagcagcttgcgattcctaactgggatcaacctatgcgcttacctggctacatgtctaggttgcctcagcctccggaagtttctcagttaggtcagatcgttaggttgatctcggagtctaagaggcctgttttgtacgttggtggtggaagcttgaactcgagtgaagaactggggagatttgtcgagcttactgggatccccgttgcgagtactttgatggggcttggctcttatccttgtaacgatgagttgtccctgcagatgcttggcatgcacgggactgtgtatgctaactacgctgtggagcatagtgatttgttgctggcgtttggtgttaggtttgatgaccgtgtcacgggaaagctcgaggctttcgctagcagggctaaaattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttccctttgagcttcaaaacgtttggagaagccattcctccgcagtacgcgattcagatcctcgacgagctaaccgaagggaaggcaattatcagtactggtgttggacagcatcagatgtgggcggcgcagttttacaagtacaggaagccgagacagtggctgtcgtcatcaggcctcggagctatgggttttggacttcctgctgcgattggagcgtctgtggcgaaccctgatgcgattgttgtggatattgacggtgatggaagcttcataatgaacgttcaagagctggccacaatccgtgtagagaatcttcctgtgaagatactcttgttaaacaaccagcatcttgggatggtcatgcaatgggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacaccaggaccatacctgttggatgtgatatgtccgcaccaagaacatgtgttaccgatgatcccaagtggtggcactttcaaagatgtaataacagaaggggatggtcgcactaagtactgagagatgaagctggtgatcgatcatatggtaaaagacttagtttcagtttccagtttcttttgtgtggtaatttgggtttgtcagttgttgtactacttttggttgttcccagacgtactcgctgttgttgttttgtttcctttttcttttatatataaataaactgcttgggttttttttcatatgtttgggactcaatgcaaggaatgctactagactgcgattatctactaatcttgctaggaaat

SEQ ID NO: 2 - protein, 655 aa, Brassica napus
MAAATSSSPISLTAKPSSKSPLPISRFSLPFSLTPQKDSSRLHRPLAISAVLNSPVNVAPPSPEKTDKNKTFVSRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSEELGRFVELTGIPVASTLMGLGSYPCNDELSLQMLGMHGTVYANYAVEHSDLLLAFGVRFDDRVTGKLEAFASRAKIVHIDIDSAEIGKNKTPHVSVCGDVKLALQGMNKVLENRAEELKLDFGVWRSELSEQKQKFPLSFKTFGEAIPPQYAIQILDELTEGKAIISTGVGQHQMWAAQFYKYRKPRQWLSSSGLGAMGFGLPAAIGASVANPDAIVVDIDGDGSFIMNVQELATIRVENLPVKILLLNNQHLGMVMQWEDRFYKANRAHTYLGDPARENEIFPNMLQFAGACGIPAARVTKKEELREAIQTMLDTPGPYLLDVICPHQEHVLPMIPSGGTFKDVITEGDGRTKY

SEQ ID NO: 3 - DNA, 3386 bp, Brassica napus
acaaccaactgaagataaaataaaattaacacaaaattatatctttcataaaaaccgatacatcaaattccgcgcgtagcggggaccctccctagtaattaatacagtaaagaaaagaccaaacaaacaaaaatcatattccaagggtattttcgtaaacaaacaaaaccctcacaagcctcgttttataaaaacgattcacgttcacaaactcattcatcatctctctctcatttctctctctctctcatctaaccatggcggcggcaacatcgtcttctccgatctccttaaccgctaaaccttcttccaaatcccctctacccatttccagattctcccttcccttctccttaaccccacagaaaccctcctcccgtctccaccgtccactcgccatctccgccgttctcaactcacccgtcaatgtcgcacctgaaaaaaccgacaagatcaagactttcatctcccgctacgctcccgacgagccccgcaagggtgctgatatcctcgtggaagccctcgagcgtcaaggcgtcgaaaccgtcttcgcttatcccggaggtgcctccatggagatccaccaagccttgactcgctcctccaccatccgtaacgtcctcccccgtcacgaacaaggaggagtcttcgccgccgagggttacgctcgttcctccggcaaaccgggaatctgcatagccacttcgggtcccggagctaccaacctcgtcagcgggttagccgacgcgatgcttgacagtgttcctctcgtcgccatcacaggacaggtccctcgccggatgatcggtactgacgcgttccaagagacgccaatcgttgaggtaacgaggtctattacgaaacataactatctggtgatggatgttgatgacatacctaggatcgttcaagaagcattctttctagctacttccggtagacccggaccggttttggttgatgttcctaaggatattcagcagcagcttgcgattcctaactgggatcaacctatgcgcttgcctggctacatgtctaggctgcctcagccaccggaagtttctcagttaggccagatcgttaggttgatctcggagtctaagaggcctgttttgtacgttggtggtggaagcttgaactcgagtgaagaactggggagatttgtcgagcttactgggatccctgttgcgagtacgttgatggggcttggctcttatccttgtaacgatgagttgtccctgcagatgcttggcatgcacgggactgtgtatgctaactacgctgtggagcatagtgatttgttgctggcgtttggtgttaggtttgatgaccgtgtcacgggaaagctcgaggcgtttgcgagcagggctaagattgtgcacatagacattgattctgctgagattgggaagaataagacacctcacgtgtctgtgtgtggtgatgtaaagctggctttgcaagggatgaacaaggttcttgagaaccgggcggaggagctcaagcttgatttcggtgtttggaggagtgagttgagcgagcagaaacagaagttcccgttgagcttcaaaacgtttggagaagccattcctccgcagtacgcgattcaggtcctagacgagctaacccaagggaaggcaattatcagtactggtgttggacagcatcagatgtgggcggcgcagttttacaagtacaggaagccgaggcagtggctgtcgtcctcaggactcggagctatgggtttcggacttcctgctgcgattggagcgtctgtggcgaaccctgatgcgattgttgtggacattgacggtgatggaagcttcataatgaacgttcaagagctggccacaatccgtgtagagaatcttcctgtgaagatactcttgttaaacaaccagcatcttgggatggtcatgcaatgggaagatcggttctacaaagctaacagagctcacacttatctcggggacccggcaagggagaacgagatcttccctaacatgctgcagtttgcaggagcttgcgggattccagctgcgagagtgacgaagaaagaagaactccgagaagctattcagacaatgctggatacacctggaccgtacctgttggatgtcatctgtccgcaccaagaacatgtgttaccgatgatcccaagtggtggcactttcaaagatgtaataaccgaaggggatggtcgcactaagtactgagagatgaagctggtgatccatcatatggtaaaagacttagtttcagttttcagtttcttttgtgtggtaatttgggtttgtcagttgttgtactgcttttggtttgttcccagacttactcgctgttgttgttttgtttcctttttcttttatatataaataaactgcttgggtttttttacataatgtttgggactcaatgcaaggaaatgctactagactgcgattatctactaatcttgcaaggaaattgcactttacattgggtgtgattgtcatgttcaatgaatcggatttagattaggacttagtttacaaagatgccagtttcactgtgttcatacctcatattttcagttttgtagccgatgaaggttcagatttcacacactatattttatatgtttgatcgatgttgtatgctcttaaacatttctatctttcgttttatcatattatccatcacactctttaatatgatgtaaattgcttcttctctaagctacaccagtcgtaagcaacatacgaagaagctttttgaaaacatcttttattctctcatcacactttgtgcatgtgtcagtaacatgtcaacttcatctgtgttgaaataaacattcggttcattgtaacaacaataatttctgtccttttttttatcaacacaataatttttctgtcttacacctatgtaacacatgttcttttctttcattctccccatttttatttaagggcgacggttcaaacccattaacagatcaaacaaccaaaccacatacagcagcttcacatcagataaacgaaggaatgcattcttgacgtcaagctgatatatgggccaatctctcacaaaacttacatcaagaacatcacgaattgtagccggtttcaccactgttgcgaaagtgtctatgtagtctatgccttcctcttgagactttccatttgcaaccaggtgtgctctatggcgtttaggcttgccttctgcatcaagcttatgtttatagagccacatacaacaaataatattagtattagtaggctttggagcaagatcccaagtcttagccttatcaaaagcatccatttctacagtcattgcaggtgtccagtttggatcttttaaagcatttatatggctagtaggcaaaggtgaaacagaagaaa

SEQ ID NO: 4 - protein, 652 aa, Brassica napus
MAAATSSSPISLTAKPSSKSPLPISRFSLPFSLTPQKPSSRLHRPLAISAVLNSPVNVAPEKTDKIKTFISRYAPDEPRKGADILVEALERQGVETVFAYPGGASMEIHQALTRSSTIRNVLPRHEQGGVFAAEGYARSSGKPGICIATSGPGATNLVSGLADAMLDSVPLVAITGQVPRRMIGTDAFQETPIVEVTRSITKHNYLVMDVDDIPRIVQEAFFLATSGRPGPVLVDVPKDIQQQLAIPNWDQPMRLPGYMSRLPQPPEVSQLGQIVRLISESKRPVLYVGGGSLNSSEELGRFVELTGIPVASTLMGLGSYPCNDELSLQMLGMHGTVYANYAVEHSDLLLAFGVRFDDRVTGKLEAFASRAKIVHIDIDSAEIGKNKTPHVSVCGDVKLALQGMNKVLENRAEELKLDFGVWRSELSEQKQKFPLSFKTFGEAIPPQYAIQVLDELTQGKAIISTGVGQHQMWAAQFYKYRKPRQWLSSSGLGAMGFGLPAAIGASVANPDAIVVDIDGDGSFIMNVQELATIRVENLPVKILLLNNQHLGMVMQWEDRFYKANRAHTYLGDPARENEIFPNMLQFAGACGIPAARVTKKEELREAIQTMLDTPGPYLLDVICPHQEHVLPMIPSGGTFKDVITEGDGRTKY

SEQ ID NO: 5 - protein, 9 aa, Artificial Sequence
  synthetic peptide
LAGLIDADG

(19) United States
(12) Patent Application Publication
(10) Pub. No.: US 2016/0138040 A1
(43) Pub. Date: May 19, 2016

(54) BRASSICA ENGINEERED TO CONFER HERBICIDE TOLERANCE

(71) Applicant: CELLECTIS, Paris (FR)

(72) Inventors: Daniel F. Voytas, Falcon Heights, MN (US); Thomas Stoddard, St. Louis Park, MN (US); Feng Zhang, Plymouth, MN (US); Luc Mathis, Le Kremlin Bicetre (FR)

(21) Appl. No.: 14/941,034

(22) Filed: Nov. 13, 2015

Related U.S. Application Data
(60) Provisional application No. 62/079,296, filed on Nov. 13, 2014.

(51) Int. Cl. C12N 15/82

(52) U.S. Cl. CPC C12N 15/8274; C12N 15/8216

(57) ABSTRACT

Materials and methods for making plants (e.g., Brassica varieties) with tolerance to ALS-inhibiting herbicides are provided herein. The methods can include making mutations in the gene encoding acetolactate synthase (ALS)/acetohydroxyacid synthase (AHAS), where the mutations are induced using a rare-cutting endonuclease and a donor matrix.

### CROSS-REFERENCE TO RELATED APPLICATIONS

This application claims benefit of priority from U.S. Provisional Application No. 62/079,296, filed on Nov. 13, 2014.

### TECHNICAL FIELD

This document provides materials and methods for creating canola varieties with herbicide tolerance.

### BACKGROUND

The U.S Food and Drug Administration granted “Generally Recognized as Safe” (GRAS) status to canola in 1985. The demand for canola has risen since then, and it is expected to continue to rise. The increased demand is likely due to the potential health benefits of canola oil. The low saturated fat and high monounsaturated fats found in canola oil have been shown to play a major role in decreasing the risk of coronary heart disease by lowering low-density lipoprotein cholesterol and serum cholesterol levels (Brown et al., U.S. Canola Association Canola Growers' Manual, University of Idaho & Oregon State University, 2008). Before the registration and release of herbicide-tolerant canola (HTC) varieties, weeds often were the limiting factor in canola production.

Beyond a reduction in yield, hard to control weeds also lead to a reduction in oil and meal quality by contaminating the harvest (Canola council of Canada: Growers Manual, available online at canolacounclorg/crop-production/canola-grower %27s-manual-contents/chapter-10a-weeds/chapter-10a##weedsofcanola).

Acetolactate synthase (ALS), also referred to as acetohydroxyacid synthase (AHAS), is responsible for the first step in the biosynthetic pathways of three essential branched-chain amino acids: isoleucine, leucine and valine. ALS is the target for multiple structurally unrelated herbicides, including sulfonylureas, imidazolinones, triazolopyrimidine sulfonamides, pyrimidinylsalicylates and sulfonylaminocarbonyl-triazolinones. These compounds are capable of inhibiting plant growth by inactivating the ALS enzyme, which is essential for amino acid biosynthesis. The advent of ALS-inhibiting herbicides has allowed farmers to help meet the rising global demand for canola. These herbicides are widely used in agriculture due to the unique mode of action that enables high weed control efficiency, high crop-weed selectivity, and low levels of mammalian toxicity (Sharper and Sing, Herbicide Activity: Toxicol Biochem Mol Biol, 69-110, 1997).

### SUMMARY

The disclosure herein is based at least in part on the discovery that canola having a modified ALS gene can be created via gene targeting, using a sequence-specific nuclease to make a targeted double-strand break (DSB) and a donor matrix to introduce the intended gene edits via homologous recombination (HR). The modified canola can have tolerance to several distinct classes of herbicides, as compared to non-modified canola. Thus, this document is based at least in part on the development of canola cultivars with increased tolerance to ALS-inhibiting herbicides, where the cultivars are created with sequence-specific nucleases and repair templates.

This document therefore provides materials and methods for creating canola varieties that have site specific ALS mutations that confer tolerance to specific classes of herbicides by disrupting herbicide mechanisms of action, or that have gain-of-function ALS mutations. Canola varieties having such modified ALS genes also are provided.

In one aspect, this document features a Brassica plant, plant part, or plant cell having at least one nucleotide mutation in at least one ALS allele endogenous to the plant, plant part, or plant cell, such that the plant, plant part, or plant cell has increased tolerance to ALS-inhibiting herbicides as compared to a control Brassica plant, plant part, or plant cell that does not have the at least one nucleotide mutation. The Brassica plant, plant part, or plant cell can be a B. napus plant, plant part, or plant cell. Each mutation can be a substitution of at least one nucleotide base pair. The plant, plant part, or plant cell can have been made using a rare-cutting endonuclease (e.g., a transcription activator-like effector endonuclease (TALE nuclease), a zinc-finger nuclease, a meganuclease, or a programmable RNA-guided endonuclease). Each of the at least one ALS allele can exhibit substitution of at least one endogenous nucleic acid, without including any exogenous nucleic acid. In some embodiments, every endogenous ALS allele can be modified. For example, every endogenous ALS allele can exhibit substitution of at least one endogenous nucleic acid, without including any exogenous nucleic acid. The plant, plant part, or plant cell can exhibit increased tolerance to ALS-inhibiting herbicides as compared to a control plant, plant part, or plant cell that lacks the mutation.

In another aspect, this document features a method for generating a Brassica plant, plant part, or plant cell having at least one nucleotide mutation in at least one ALS allele endogenous to the plant, plant part, or plant cell, such that the plant, plant part, or plant cell has increased tolerance to ALS-inhibiting herbicides as compared to a control Brassica plant, plant part, or plant cell that does not have the at least one nucleotide mutation. The method can include (a) contacting a Brassica plant, plant part, or plant cell having an endogenous ALS gene that can be modified to confer tolerance, with a rare-cutting endonuclease targeted to the endogenous ALS gene; (b) contacting the Brassica plant, plant part, or plant cell with a donor matrix containing a sequence homologous to the endogenous ALS gene, with the exception of the at least one nucleotide mutation; and (c) growing the selected plant part or plant cell into a Brassica plant, wherein the Brassica plant has increased tolerance to ALS-inhibiting herbicides as compared to the control Brassica plant, to produce a Brassica plant, plant part, or plant cell having tolerance to ALS-inhibiting herbicides, in which homologous recombination of the donor matrix has conferred the tolerance. The Brassica plant cells can be protoplasts. The rare-cutting endonuclease can be encoded by a nucleic acid (e.g., an mRNA), which can be contained within a vector (e.g., a viral vector). The rare-cutting endonuclease can be a protein. The rare-cutting endonuclease can be a TALE nuclease, a zinc-finger nuclease, a meganuclease, or a programmable RNA-guided endonuclease. The donor matrix can include a single-stranded nucleic acid (e.g., a ribonucleic acid). The donor matrix can be contained within a vector (e.g., a viral vector). The method can further include culturing the protoplasts to generate plant lines. The method can include isolating genomic DNA containing at least a portion of the ALS loci from the protoplasts. The Brassica plant cells can be B. napus plant cells.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DETAILED DESCRIPTION

This document provides canola plant varieties, particularly of the species Brassica napus, having modified ALS amino-acid sequences that confer tolerance to ALS-inhibiting herbicides. Methods for generating such plant varieties are provided.

As used herein, the terms “plant” and “plant part” refer to cells, tissues, organs, seeds, and severed parts (e.g., roots, leaves, and flowers) that retain the distinguishing characteristics of the parent plant. “Seed” refers to any plant structure that is formed by continued differentiation of the ovule of the plant, following its normal maturation point at flower opening, irrespective of whether it is formed in the presence or absence of fertilization and irrespective of whether or not the seed structure is fertile or infertile.

The term “allele(s)” means any of one or more alternative forms of a gene at a particular locus. In a diploid (or amphidiploid) cell of an organism, alleles of a given gene are located at a specific location or locus on a chromosome, with one allele being present on each chromosome of the pair of homologous chromosomes. “Heterozygous” alleles are different alleles residing at a specific locus, positioned individually on corresponding homologous chromosomes. “Homozygous” alleles are identical alleles residing at a specific locus, positioned individually on corresponding homologous chromosomes in the cell.

“Wild type” as used herein refers to a typical form of a plant or a gene as it most commonly occurs in nature. A “wild type ALS allele” is a naturally occurring ALS allele (e.g., as found within naturally occurring B. napus plants) that encodes a functional ALS protein.

“Modified mutant ALS allele” as used herein refers to an ALS allele that encodes a functional ALS protein having a modified amino acid sequence as compared to a wild type ALS allele, such that it is tolerant to several structurally distinct herbicides, sulfonylureas, imidazolinones, triazolopyrimidine sulfonamides, pyrimidinylsalicylates and sulfonylaminocarbonyl-triazolinones. Such a modified mutant ALS allele can include one or more mutations in its nucleic acid sequence, where the mutation(s) result in a reduced or even no detectable sensitivity to the aforementioned herbicides in the plant or plant cell in vivo. In some embodiments, a modified mutant ALS allele can encode an ALS protein having one or more amino acid substitutions disclosed in U.S. Publication No. 2014/0135219. For example, a modified mutant ALS allele can have an amino acid substitution within an ALS I and/or ALS III protein corresponding to alanine 190 (A190) of ALS I or alanine 187 (A187) of ALS III, tryptophan 559 (W559) of ALS I or tryptophan 556 (W556) of ALS III, serine 638 (S638) of ALS I or serine 5635 (S635) of ALS III, and/or glycine 639 (G639) of ALS I or glycine 636 (G636) of ALS III. A representative ALS I nucleotide sequence from B. napus is set forth in SEQ ID NO:1:

(SEQ ID NO: 1)
TCTCTAGATNCGGANCCATTTTCTCAGATNCTCTTATCTTTTTCTTCTTG
TTTCTAGGTTATCTTGAAGCCACTTTCTCCTCTTTGTCTCTATAAGCAAA
GCAATAGCATACATAACACAATCAATCACTCATCTTCCTATACCTTATTA
CAAAACTAAAGCATGAAGCTTCCTCGTAACATAGACTTCACTAACATTGC
TTACTGCAGTTGTAACGTGAGTATTGTCCGCCTGAGATCCTTCTCTTGTA
AGCCTATGGTCCCATATTTTCTGAAAAATAATTAGTTAACAGCACGTTAG
AAGATTTGAATATCACCTTCCCAACCAAATATATTACACACTATCATCCA
TTGAACCACATATTAACCAAACATTGGCTGTTAGAGAATCCAGCACACAA
ATTTCTCCCCAAGATATCGCAATACAGTTCACAGAAGTCAGACTCCTAGT
CTAATGTTCGTGGGCCATAGTCGAACATATATATACAGTAAGTGTGTTAA
GTTGTTCCTTCATTAACTTTCAAGAACTGTCATGATCAGAATCGACCAAG
TTTCCTATAAAGTGGTTTTTAGGTTTTAAGATTTTACCTTAACTTAATGT
AAASSAGACAATACTTCAGTGGCACCAAGCATGATCCAAAGGCTTTGTTT
CCATGGTAACGTGCAAGCTCACCGAAGGGGTCTCACTGAAGAAGCTGTAC
CTCGTCCTCGTCTTCTTTTCCGGCCTCTAACTGAAATGGAATATCCAGTG
GTCGCTTATATATGGTTGAAAAACAAATGAGTGAATTATCTATTCCAGCA
CAAAGCAAGTATTTGAACCATATGGTCAACGTGCGTAATGTTCTTACGTT
GGAAGATCTTGCTTGGTTAGTTTGGGATAAGTAACAATGCAGCATCAAAG
TCTCCTTGACAATCTAAGGCTTTCCATATAGTTCAAGACACCAGAGAAGC
TGGGAACTTTTCTTGGAGGTCAGACATATTGCTCGCCAAAGCTTTGTGGA
GTACCCCATCATCAGAAAGCGCAATCCAAGCACCAAAATCATCAGTAGCA
CTCGCCGTTCTTCTTGAAGCTGATTTACTTTCACACCATTTGTGGAAATA
TGTTTCCTTTAAGAGTCAGATGGTCCAGCTAAGAAAAGGGTAAGCTCATT
ACGATCATCTTGATCATCTTCGTCCAAACGAACCCGTCTCGGAGCTCCTT
TGCTAGCTTCTTTGTGAGACTTCTCTATCTTCACAACCTGAGTCTCCATA
GCGAAAGTAAACTCACGATGCCTGTCTCGAGTTTCGTCTTTCAAACCATT
GTCGTAGCTAGATTGAACATCATTTTGGAACTCATCTAAGTACAGAGAAA
CGATACTGGCAATCAATAAACGTTATACAATAAGACAATATGAGAGCAGA
GAAGGAGATAAAGAATGAAACCTGCCATTTGGTGGTTCCAGAGAGCGTCG
TGAAGGTCCCTGTGGAGCTGATCTTACCGACCGAACCTCTTCGCCATCTC
CTGAGATTTTTAGAAAATGGAAAGAAGGGAAGCAAAAGAGAATAATCATG
GATTTGAATAACTGGAAACAATAAAGTAATAAAAGATTCAAGGAATCGCT
TAATCAGAAATCAAAGTGTAAAAAAAAATTAGAAACCATAAAGTAATAAT
AGGAAATCTCCAATTAATTTGCCAAGACATATCACCTGCGATGTATTCAT
ACACATTCCGAGTGATAGAGAAGGAACCGTTTCCGTTGATAGAAGAGAAG
GCAAAACGTCAAGATTGATGATCTACAAAGGAAATCATCACGTGAATTGA
AATCTCGAAGTTCCATAGCTGGTTACAGAGATCGAAGGAGAAGCCTTTTC
GATTCAGTGTGGAAGAAGAACAGGAACTCTAAAGTTTGTGCTTTGGTCTT
GCTGGCCAAACTCAGAGCCCAACGTAATAACCAACGGAAGCCCATCAACA
TTTGCGTTTAATGAAACCATGAGTTTCGTTCCTGAATGACATGTGTCAGC
ACGGGGGAAATCCACTTATCTATATAAAGTTTCACGCTCAAGTTGAACGA
CTTAGTAACATGGAAGCTGAGGTAGATAGTCTAAGAGTGAAGAAAATTCT
TCTTTATAATAATAGATATTGTAGCCTAAACGTTCTTAGTGCACATTTTT
AAATGCAAATATATTACGGATTTGGTCCTAATTTCTGTAAAATAAAACTA
CTTCATTGTTTTATATAAATAACTCTATTGCTGCAGTTTCCCAACTTTGT
TGCTTAGATTCAGGTCTCACAATCAAGAAATCAAGAACAGTTAGATCCAC
AAATTCTACATTTTATTTAATAAGTGAAGTGACAAAAAACGAGATTAGAT
TCGTTTCTATTCATCCATAATTAATAAAAAAAAAAGACCAAACAAACAAA
AATCATATTCCAAGGGTATTTTCGTAAACAAACAAAACCCTCACAAGTCT
CGTTTTATAAAACGATTCACGTTCACAAACTCATTCATCATCTCTCTCTC
CTCTAACCATGGCGGCGGCAACATCGTCTTCTCCGATCTCCTTAACCGCT
AAACCTTCTTCCAAATCCCCTCTACCCATTTCCAGATTCTCCCTTCCCTT
CTCCTTAACCCCACAGAAAGACTCCTCCCGTCTCCACCGTCCTCTCGCCA
TCTCCGCCGTTCTCAACTCACCCGTCAATGTCGCACCTCCTTCCCCTGAA
AAAACCGACAAGAACAAGACTTTCGTCTCCCGCTACGCTCCCGACGAGCC
CCGCAAGGGTGCTGATATCCTCGTCGAAGCCCTCGAGCGTCAAGGCGTCG
AAACCGTCTTTGCTTATCCCGGAGGTGCTTCCATGGAGATCCACCAAGCC
TTGACTCGCTCCTCCACCATCCGTAACGTCCTTCCCCGTCACGAACAAGG
AGGAGTCTTCGCCGCCGAGGGTTACGCTCGTTCCTCCGGCAAACCGGGAA
TCTGCATAGCCACTTCGGGTCCCGGAGCTACCAACCTCGTCAGCGGGTTA
GCAGACGCGATGCTTGACAGTGTTCCTCTTGTCGCCATTACAGGACAGGT
CCCTCGCCGGATGATCGGTACTGACGCCTTCCAAGAGACACCAATCGTTG
AGGTAACGAGGTCTATTACGAAACATAACTATTTGGTGATGGATGTTGAT
GACATACCTAGGATCGTTCAAGAAGCTTTCTTTCTAGCTACTTCCGGTAG
ACCCGGACCGGTTTTGGTTGATGTTCCTAAGGATATTCAGCAGCAGCTTG
CGATTCCTAACTGGGATCAACCTATGCGCTTACCTGGCTACATGTCTAGG
TTGCCTCAGCCTCCGGAAGTTTCTCAGTTAGGTCAGATCGTTAGGTTGAT
CTCGGAGTCTAAGAGGCCTGTTTTGTACGTTGGTGGTGGAAGCTTGAACT
CGAGTGAAGAACTGGGGAGATTTGTCGAGCTTACTGGGATCCCCGTTGCG
AGTACTTTGATGGGGCTTGGCTCTTATCCTTGTAACGATGAGTTGTCCCT
GCAGATGCTTGGCATGCACGGGACTGTGTATGCTAACTACGCTGTGGAGC
ATAGTGATTTGTTGCTGGCGTTTGGTGTTAGGTTTGATGACCGTGTCACG
GGAAAGCTCGAGGCTTTCGCTAGCAGGGCTAAAATTGTGCACATAGACAT
TGATTCTGCTGAGATTGGGAAGAATAAGACACCTCACGTGTCTGTGTGTG
GTGATGTAAAGCTGGCTTTGCAAGGGATGAACAAGGTTCTTGAGAACCGG
GCGGAGGAGCTCAAGCTTGATTTCGGTGTTTGGAGGAGTGAGTTGAGCGA
GCAGAAACAGAAGTTCCCTTTGAGCTTCAAAACGTTTGGAGAAGCCATTC
CTCCGCAGTACGCGATTCAGATCCTCGACGAGCTAACCGAAGGGAAGGCA
ATTATCAGTACTGGTGTTGGACAGCATCAGATGTGGGCGGCGCAGTTTTA
CAAGTACAGGAAGCCGAGACAGTGGCTGTCGTCATCAGGCCTCGGAGCTA
TGGGTTTTGGACTTCCTGCTGCGATTGGAGCGTCTGTGGCGAACCCTGAT
GCGATTGTTGTGGATATTGACGGTGATGGAAGCTTCATAATGAACGTTCA
AGAGCTGGCCACAATCCGTGTAGAGAATCTTCCTGTGAAGATACTCTTGT
TAAACAACCAGCATCTTGGGATGGTCATGCAATGGGAAGATCGGTTCTAC
AAAGCTAACAGAGCTCACACTTATCTCGGGGACCCGGCAAGGGAGAACGA
GATCTTCCCTAACATGCTGCAGTTTGCAGGAGCTTGCGGGATTCCAGCTG
CGAGAGTGACGAAGAAAGAAGAACTCCGAGAAGCTATTCAGACAATGCTG
GATACACCAGGACCATACCTGTTGGATGTGATATGTCCGCACCAAGAACA
TGTGTTACCGATGATCCCAAGTGGTGGCACTTTCAAAGATGTAATAACAG
AAGGGGATGGTCGCACTAAGTACTGAGAGATGAAGCTGGTGATCGATCAT
ATGGTAAAAGACTTAGTTTCAGTTTCCAGTTTCTTTTGTGTGGTAATTTG
GGTTTGTCAGTTGTTGTACTACTTTTGGTTGTTCCCAGACGTACTCGCTG
TTGTTGTTTTGTTTCCTTTTTCTTTTATATATAAATAAACTGCTTGGGTT
TTTTTTCATATGTTTGGGACTCAATGCAAGGAATGCTACTAGACTGCGAT
TATCTACTAATCTTGCTAGGAAAT.

A representative ALS1 polypeptide sequence translated from SEQ ID NO:1 is set forth in SEQ ID NO:2:

(SEQ ID NO: 2)
MAAATSSSPISLTAKPSSKSPLPISRFSLPFSLTPQKDSSRLHRPLAISA
VLNSPVNVAPPSPEKTDKNKTFVSRYAPDEPRKGADILVEALERQGVETV
FAYPGGASMEIHQALTRSSTIRNVLPRHEQGGVFAAEGYARSSGKPGICI
ATSGPGATNLVSGLADAMLDSVPLVAITGQVPRRMIGTDAFQETPIVEVT
RSITKHNYLVMDVDDIPRIVQEAFFLATSGRPGPVLVDVPKDIQQQLAIP
NWDQPMRLPGYMSRLPQPPEVSQLGQIVRLISESKRPVLYVGGGSLNSSE
ELGRFVELTGIPVASTLMGLGSYPCNDELSLQMLGMHGTVYANYAVEHSD
LLLAFGVRFDDRVTGKLEAFASRAKIVHIDIDSAEIGKNKTPHVSVCGDV
KLALQGMNKVLENRAEELKLDFGVWRSELSEQKQKFPLSFKTFGEAIPPQ
YAIQILDELTEGKAIISTGVGQHQMWAAQFYKYRKPRQWLSSSGLGAMGF
GLPAAIGASVANPDAIVVDIDGDGSFIMNVQELATIRVENLPVKILLLNN
QHLGMVMQWEDRFYKANRAHTYLGDPARENEIFPNMLQFAGACGIPAARV
TKKEELREAIQTMLDTPGPYLLDVICPHQEHVLPMIPSGGTFKDVITEGD
GRTKY.

A representative ALS3 nucleotide sequence from B. napus is set forth in SEQ ID NO:3:

(SEQ ID NO: 3)
ACAACCAACTGAAGATAAAATAAAATTAACACAAAATTATATCTTTCAT
AAAAACCGATACATCAAATTCCGCGCGTAGCGGGGACCCTCCCTAGTAA
TTAATACAGTAAAGAAAAGACCAAACAAACAAAAATCATATTCCAAGGG
TATTTTCGTAAACAAACAAAACCCTCACAAGCCTCGTTTTATAAAAACG
ATTCACGTTCACAAACTCATTCATCATCTCTCTCTCATTTCTCTCTCTC
TCTCATCTAACCATGGCGGCGGCAACATCGTCTTCTCCGATCTCCTTAA
CCGCTAAACCTTCTTCCAAATCCCCTCTACCCATTTCCAGATTCTCCCT
TCCCTTCTCCTTAACCCCACAGAAACCCTCCTCCCGTCTCCACCGTCCA
CTCGCCATCTCCGCCGTTCTCAACTCACCCGTCAATGTCGCACCTGAAA
AAACCGACAAGATCAAGACTTTCATCTCCCGCTACGCTCCCGACGAGCC
CCGCAAGGGTGCTGATATCCTCGTGGAAGCCCTCGAGCGTCAAGGCGTC
GAAACCGTCTTCGCTTATCCCGGAGGTGCCTCCATGGAGATCCACCAAG
CCTTGACTCGCTCCTCCACCATCCGTAACGTCCTCCCCCGTCACGAACA
AGGAGGAGTCTTCGCCGCCGAGGGTTACGCTCGTTCCTCCGGCAAACCG
GGAATCTGCATAGCCACTTCGGGTCCCGGAGCTACCAACCTCGTCAGCG
GGTTAGCCGACGCGATGCTTGACAGTGTTCCTCTCGTCGCCATCACAGG
ACAGGTCCCTCGCCGGATGATCGGTACTGACGCGTTCCAAGAGACGCCA
ATCGTTGAGGTAACGAGGTCTATTACGAAACATAACTATCTGGTGATGG
ATGTTGATGACATACCTAGGATCGTTCAAGAAGCATTCTTTCTAGCTAC
TTCCGGTAGACCCGGACCGGTTTTGGTTGATGTTCCTAAGGATATTCAG
CAGCAGCTTGCGATTCCTAACTGGGATCAACCTATGCGCTTGCCTGGCT
ACATGTCTAGGCTGCCTCAGCCACCGGAAGTTTCTCAGTTAGGCCAGAT
CGTTAGGTTGATCTCGGAGTCTAAGAGGCCTGTTTTGTACGTTGGTGGT
GGAAGCTTGAACTCGAGTGAAGAACTGGGGAGATTTGTCGAGCTTACTG
GGATCCCTGTTGCGAGTACGTTGATGGGGCTTGGCTCTTATCCTTGTAA
CGATGAGTTGTCCCTGCAGATGCTTGGCATGCACGGGACTGTGTATGCT
AACTACGCTGTGGAGCATAGTGATTTGTTGCTGGCGTTTGGTGTTAGGT
TTGATGACCGTGTCACGGGAAAGCTCGAGGCGTTTGCGAGCAGGGCTAA
GATTGTGCACATAGACATTGATTCTGCTGAGATTGGGAAGAATAAGACA
CCTCACGTGTCTGTGTGTGGTGATGTAAAGCTGGCTTTGCAAGGGATGA
ACAAGGTTCTTGAGAACCGGGCGGAGGAGCTCAAGCTTGATTTCGGTGT
TTGGAGGAGTGAGTTGAGCGAGCAGAAACAGAAGTTCCCGTTGAGCTTC
AAAACGTTTGGAGAAGCCATTCCTCCGCAGTACGCGATTCAGGTCCTAG
ACGAGCTAACCCAAGGGAAGGCAATTATCAGTACTGGTGTTGGACAGCA
TCAGATGTGGGCGGCGCAGTTTTACAAGTACAGGAAGCCGAGGCAGTGG
CTGTCGTCCTCAGGACTCGGAGCTATGGGTTTCGGACTTCCTGCTGCGA
TTGGAGCGTCTGTGGCGAACCCTGATGCGATTGTTGTGGACATTGACGG
TGATGGAAGCTTCATAATGAACGTTCAAGAGCTGGCCACAATCCGTGTA
GAGAATCTTCCTGTGAAGATACTCTTGTTAAACAACCAGCATCTTGGGA
TGGTCATGCAATGGGAAGATCGGTTCTACAAAGCTAACAGAGCTCACAC
TTATCTCGGGGACCCGGCAAGGGAGAACGAGATCTTCCCTAACATGCTG
CAGTTTGCAGGAGCTTGCGGGATTCCAGCTGCGAGAGTGACGAAGAAAG
AAGAACTCCGAGAAGCTATTCAGACAATGCTGGATACACCTGGACCGTA
CCTGTTGGATGTCATCTGTCCGCACCAAGAACATGTGTTACCGATGATC
CCAAGTGGTGGCACTTTCAAAGATGTAATAACCGAAGGGGATGGTCGCA
CTAAGTACTGAGAGATGAAGCTGGTGATCCATCATATGGTAAAAGACTT
AGTTTCAGTTTTCAGTTTCTTTTGTGTGGTAATTTGGGTTTGTCAGTTG
TTGTACTGCTTTTGGTTTGTTCCCAGACTTACTCGCTGTTGTTGTTTTG
TTTCCTTTTTCTTTTATATATAAATAAACTGCTTGGGTTTTTTTACATA
ATGTTTGGGACTCAATGCAAGGAAATGCTACTAGACTGCGATTATCTAC
TAATCTTGCAAGGAAATTGCACTTTACATTGGGTGTGATTGTCATGTTC
AATGAATCGGATTTAGATTAGGACTTAGTTTACAAAGATGCCAGTTTCA
CTGTGTTCATACCTCATATTTTCAGTTTTGTAGCCGATGAAGGTTCAGA
TTTCACACACTATATTTTATATGTTTGATCGATGTTGTATGCTCTTAAA
CATTTCTATCTTTCGTTTTATCATATTATCCATCACACTCTTTAATATG
ATGTAAATTGCTTCTTCTCTAAGCTACACCAGTCGTAAGCAACATACGA
AGAAGCTTTTTGAAAACATCTTTTATTCTCTCATCACACTTTGTGCATG
TGTCAGTAACATGTCAACTTCATCTGTGTTGAAATAAACATTCGGTTCA
TTGTAACAACAATAATTTCTGTCCTTTTTTTTATCAACACAATAATTTT
TCTGTCTTACACCTATGTAACACATGTTCTTTTCTTTCATTCTCCCCAT
TTTTATTTAAGGGCGACGGTTCAAACCCATTAACAGATCAAACAACCAA
ACCACATACAGCAGCTTCACATCAGATAAACGAAGGAATGCATTCTTGA
CGTCAAGCTGATATATGGGCCAATCTCTCACAAAACTTACATCAAGAAC
ATCACGAATTGTAGCCGGTTTCACCACTGTTGCGAAAGTGTCTATGTAG
TCTATGCCTTCCTCTTGAGACTTTCCATTTGCAACCAGGTGTGCTCTAT
GGCGTTTAGGCTTGCCTTCTGCATCAAGCTTATGTTTATAGAGCCACAT
ACAACAAATAATATTAGTATTAGTAGGCTTTGGAGCAAGATCCCAAGTC
TTAGCCTTATCAAAAGCATCCATTTCTACAGTCATTGCAGGTGTCCAGT
TTGGATCTTTTAAAGCATTTATATGGCTAGTAGGCAAAGGTGAAACAGA
AGAAA.

A representative ALS3 polypeptide sequence translated from SEQ ID NO:3 is set forth in SEQ ID NO:4:

(SEQ ID NO: 4)
MAAATSSSPISLTAKPSSKSPLPISRFSLPFSLTPQKPSSRLHRPLAISA
VLNSPVNVAPEKTDKIKTFISRYAPDEPRKGADILVEALERQGVETVFAY
PGGASMEIHQALTRSSTIRNVLPRHEQGGVFAAEGYARSSGKPGICIATS
GPGATNLVSGLADAMLDSVPLVAITGQVPRRMIGTDAFQETPIVEVTRSI
TKHNYLVMDVDDIPRIVQEAFFLATSGRPGPVLVDVPKDIQQQLAIPNWD
QPMRLPGYMSRLPQPPEVSQLGQIVRLISESKRPVLYVGGGSLNSSEELG
RFVELTGIPVASTLMGLGSYPCNDELSLQMLGMHGTVYANYAVEHSDLLL
AFGVRFDDRVTGKLEAFASRAKIVHIDIDSAEIGKNKTPHVSVCGDVKLA
LQGMNKVLENRAEELKLDFGVWRSELSEQKQKFPLSFKTFGEAIPPQYAI
QVLDELTQGKAIISTGVGQHQMWAAQFYKYRKPRQWLSSSGLGAMGFGLP
AAIGASVANPDAIVVDIDGDGSFIMNVQELATIRVENLPVKILLLNNQHL
GMVMQWEDRFYKANRAHTYLGDPARENEIFPNMLQFAGACGIPAARVTKK
EELREAIQTMLDTPGPYLLDVICPHQEHVLPMIPSGGTFKDVITEGDGRT
KY.

“Mutagenesis” as used herein refers to processes in which mutations are introduced into a selected DNA sequence. Mutations induced by endonucleases generally are obtained by a double strand break, which results in insertion/deletion mutations (“indels”) that can be detected by deep-sequencing analysis. Such mutations typically are deletions or insertions of one or several base pairs, and may result in inactivation of an allele if mutations occur in the allele's coding sequence, promoter sequence, or intronic splicing elements. As described in the Examples section herein, for example, mutagenesis can occur via double-stranded DNA breaks made by transcription activator-like effector nucleases (TALE nucleases) targeted to selected DNA sequences in a plant cell. Such mutagenesis results in “TALE nuclease-induced mutations” (e.g., TALE nuclease-induced knockouts) and reduced expression of the targeted gene. Following mutagenesis, plants can be regenerated from the treated cells using known techniques (e.g., planting seeds in accordance with conventional growing procedures, followed by self-pollination). In the present document, mutagenesis is not limited to punctual mutations. Any gene repair or deletion performed on the endogenous gene conferring herbicide tolerance to the plant is regarded as a mutation of the gene.

The term “expression” as used herein refers to the transcription of a particular nucleic acid sequence to produce sense or antisense RNA or mRNA, and/or the translation of an mRNA molecule to produce a polypeptide, with or without subsequent post-translational events.

The canola genome usually contains an ALS gene family of five members. The methods provided herein can be used to modify at least one (e.g., at least two, at least three, at least four, or all five) functional alleles of ALS, thereby conferring herbicide tolerance.

In some embodiments, the B. napus plants, cells, plant parts, seeds, and progeny thereof that are provided herein can have a mutation in each endogenous ALS allele, such that expression of the gene is capable of conferring tolerance to ALS-inhibiting herbicides. Thus, in some cases, the plants, cells, plant parts, seeds, and progeny are tolerant to ALS-inhibiting herbicides.

The plants, plant cells, plant parts, seeds, and progeny provided herein can be generated using a rare-cutting endonuclease (e.g., a TALE nuclease) and a “donor matrix” containing a sequence that is homologous to the endogenous gene, with the exception of the intended gene edits, to make a sequence-specific modification(s) in one or more alleles of the ALS gene. Thus, this document provides materials and methods for using rare-cutting endonucleases (e.g., TALE nucleases) and a donor matrix to generate Brassica plants and related products (e.g., seeds and plant parts) that are particularly suitable for conferring ALS-inhibiting herbicide tolerance. Other sequence-specific nucleases also can be used to generate the desired plant material, including engineered homing endonucleases, zinc finger nucleases (ZFNs), or programmable RNA-guided endonucleases.

The term “rare-cutting endonuclease” as used herein refers to a natural or engineered protein having endonuclease activity directed to a nucleic acid sequence with a recognition sequence (target sequence) about 12-40 bp in length (e.g., 14-40, 15-36, or 16-32 bp in length; see, e.g., Baker, Nature Methods 9:23-26, 2012). Typical rare-cutting endonucleases cause cleavage inside their recognition site, leaving 4 nt staggered cuts with 3′ OH or 5′ OH overhangs. In some embodiments, a rare-cutting endonuclease can be a meganuclease, such as a wild type or variant homing endonuclease (e.g., a homing endonuclease belonging to the dodecapeptide family (LAGLIDADG (SEQ ID NO:5); see, PCT Publication No. WO 2004/067736). In some embodiments, a rare-cutting endonuclease can be a programmable RNA-guided endonuclease, such as the RNA-guided Cas9 nuclease from the type II prokaryotic CRISPR (Clustered Regularly Interspaced Short palindromic Repeats) adaptive immune system (see, e.g., Belahj et al., Plant Methods 9:39, 2013).

In some embodiments, a rare-cutting endonuclease can be a fusion protein that contains a DNA binding domain and a catalytic domain with cleavage activity. TALE nucleases and ZFNs are examples of fusions of DNA binding domains with the catalytic domain of the endonuclease FokI.

Transcription activator-like (TAL) effectors are found in plant pathogenic bacteria in the genus Xanthomonas. These proteins play important roles in disease, or trigger defense, by binding host DNA and activating effector-specific host genes (see, e.g., Gu et al., Nature 435:1122-1125, 2005; Yang et al., Proc. Natl. Acad. Sci. USA 103:10503-10508, 2006; Kay et al. Science 318:648-651, 2007; Sugio et al., Proc. Natl. Acad. Sci. USA 104:10720-10725, 2007; and Römer et al. Science 318:645-648, 2007). Specificity depends on an effector-variable number of imperfect, typically 34 amino acid repeats (Schornack et al., J. Plant Physiol. 163:256-272, 2006; and WO 2011/072246). Polymorphisms are present primarily at repeat positions 12 and 13, which are referred to as the repeat variable-diresidue (RVD).

The RVDs of TAL effectors correspond to the nucleotides in their target sites in a direct, linear fashion, one RVD to one nucleotide, with some degeneracy and no apparent context dependence. This mechanism for protein-DNA recognition enables target site prediction for new target specific TAL effectors, as well as target site selection and engineering of new TAL effectors with binding specificity for the selected sites.

TAL effector DNA binding domains can be fused to other sequences, such as endonuclease sequences, resulting in chimeric endonucleases targeted to specific, selected DNA sequences, and leading to subsequent cutting of the DNA at or near the targeted sequences. Such cuts (double-stranded breaks) in DNA can induce mutations into the wild type DNA sequence via non-homologous end joining (NHEJ) or homologous recombination, for example. In some cases, TALE nucleases can be used to facilitate site directed mutagenesis in complex genomes, knocking out or otherwise altering gene function with great precision and high efficiency. TALE nucleases targeted to the B. napus ALS gene can be used to increase homologous recombination with an exogenous donor matrix to mutagenize the endogenous gene, resulting in plants with modified ALS proteins tolerant to ALS-inhibiting herbicides. The fact that some endonucleases (e.g., FokI) function as dimers can be used to enhance the target specificity of the TALE nuclease. For example, in some cases a pair of TALE nuclease monomers targeted to different DNA sequences can be used. When the two TALE nuclease recognition sites are in close proximity the inactive monomers can come together to create a functional enzyme that cleaves the DNA. By requiring DNA binding to activate the nuclease, a highly site-specific restriction enzyme can be created. Customized TALE nucleases are commercially available under the trade name TALEN™ (Cellectis, Paris, France).

The term “homologous” as used herein refers to a sequence with enough identity to a target sequence to lead to a homologous recombination between the two sequences. In some embodiments, a pair of homologous sequences can have at least 95% sequence identity (e.g., 96%, 97%, 98%, or 99% sequence identity) to one another.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100.

The term “tolerance” as used herein refers to a plant that is tolerant or resistant to at least one ALS-inhibiting herbicide. Tolerance is determined by subjecting modified mutant and wild-type plants to a range of herbicides in lethal and sub-lethal doses. The dose required to reduce shoot weight by 50% is then used to determine the resistant to susceptible (R/S) ratio. If the ratio is greater than 1, then the resistant plant is considered tolerant.

The term “herbicide” as used herein designates any chemical substance that inhibits the growth of the plant. The tolerance of a plant to an herbicide may be partial, for instance when tolerance occurs with respect to a certain concentration of the substance, or in the presence or absence of co-factors or external factors (e.g., temperature and/or humidity).

The term “nickase” as used herein refers to an endonuclease that cleaves only one of two DNA strands.

Methods for selecting endogenous target sequences and generating TALE nucleases targeted to such sequences can be performed as described elsewhere. See, for example, PCT Publication No. WO 2011/072246, which is incorporated herein by reference in its entirety. In some embodiments, software that specifically identifies TALE nuclease recognition sites, such as TALE-NT 2.0 (Doyle et al., Nucleic Acids Res 40:W117-122, 2012) can be used.

Methods for using rare-cutting endonucleases (e.g., TALE nucleases) to generate canola plants, plant cells, or plant parts having mutations in endogenous genes include, for example, those to be described in the Examples herein. For example, one or more nucleic acids encoding TALE nucleases targeted to selected ALS sequences can be transformed into plant cells (e.g., protoplasts), where they can be expressed. In some cases, one or more TALE nuclease proteins can be introduced into plant cells (e.g., protoplasts). The cells, or a plant cell line or plant part generated from the cells, can subsequently be analyzed to determine whether mutations have been introduced at the target site(s), through nucleic acid-based assays or protein-based assays to detect expression levels as described above, for example, or using nucleic acid-based assays (e.g., PCR and DNA sequencing, or PCR followed by a T7E1 assay; Mussolino et al., Nucleic Acids Res. 39:9283-9293, 2011) to detect mutations at the genomic loci. In a T7E1 assay, genomic DNA can be isolated from pooled calli, and sequences flanking TALE nuclease recognition sites for ALS can be PCR-amplified. Amplification products then can be denatured and re-annealed. If the re-annealed fragments form a heteroduplex, T7 endonuclease I cuts at the site of mismatch. The digested products can be visualized by gel electrophoresis to quantify mutagenesis activity of the TALE nuclease.

In some embodiments, a method as provided herein can include contacting a population of Brassica plant cells (e.g., protoplasts) having a functional ALS allele with a rare-cutting endonuclease that is targeted to an endogenous ALS sequence, selecting from the population a cell in which at least one (e.g., one, two, three, four, or five) ALS alleles have been modified, and growing the selected cell into a Brassica plant. The plant may have increased tolerance to ALS-inhibiting herbicides, as compared to a control Brassica plant that does not contain the modified ALS alleles. The rare-cutting endonuclease can be introduced into the population of cells via a nucleic acid (e.g., a vector or an mRNA) that encodes the rare-cutting endonuclease, or as a protein. In some cases, a method as provided herein can include a step of culturing a plant cell containing the modified ALS allele(s) to generate one or more plant lines. In addition or alternatively, a method as provided herein can include a step of isolating genomic DNA containing at least a portion of the ALS locus from the plant cells.

Another genome engineering tool that can be used in the methods provided herein is based on the RNA-guided Cas9 nuclease from the type II prokaryotic CRISPR adaptive immune system (see, e.g., Belahj et al., supra). This system allows for cleavage of DNA sequences that are adjacent to a short sequence motif, referred as proto-spacer adjacent motif (PAM). Binding of Cas9 to a target sequence is achieved by delivering Cas9 to a plant cell, along with a crRNA that is complementary to the target sequence. The crRNA base pairs with a trans-activating tracrRNA to form a guide RNA (gRNA), which then directs Cas9 to the target sequence. Once there, Cas9 generates a DNA DSB at a position three nucleotides from the 3′ end of the crRNA sequence that is complementary to the target sequence. To reduce the complexity of the system for genome engineering, the crRNA and tracrRNA can be fused to generate a single guide RNA (sgRNA). Since several PAM motifs are present in the nucleotide sequence of the ALS gene, crRNA or sgRNA specific to ALS gene can be designed to introduce a DSB to enhance gene targeting events within Brassica plant cells into which the Cas9 endonuclease and the crRNA or sgRNA are transfected and then expressed. In some embodiments, therefore, this approach can be used to obtain ALS mutant plants as described herein.

Herbicide tolerant Brassica plants, plant parts, and plant cells made by the methods described herein also are provided.

The invention will be further described in the following examples, which do not limit the scope of the invention described in the claims.

### EXAMPLES

### Example 1

### Engineering Sequence-Specific Nucleases to Mutagenize the ALS Gene

Sequence-specific nucleases are generated to create DSBs at one or more target ALS genes in B. napus to mediate homologous recombination of the donor matrices, introducing the intended gene edits. In particular, a pair of TALE nucleases is designed to target the ALS gene family, using software that specifically identifies TALE nuclease recognition sites. TALE nucleases are synthesized using methods similar to those described elsewhere (Cermak et al., Nucleic Acids Res. 39:e82, 2011; Reyon et al., Nat. Biotechnol. 30:460-465, 2012; and Zhang et al., Nat. Biotechnol. 29:149-153, 2011).

### Example 2

### ALS TALE Nuclease Activity at their Endogenous Target Sites in B. napus

TALE nuclease activity at endogenous target sites in B. napus is measured by expressing the TALE nucleases in protoplasts and subsequently surveying the target sites for mutations introduced by NHEJ. Protoplast are prepared as described elsewhere (Watanabe et al., Physiologia Plantarium 86: 231-235, 1992). Briefly, leaves from 6-8 week old plants are used to prepare the protoplasts. Young, fully expanded leaves are collected and surface sterilized, and protoplasts are isolated.

TALE nuclease-encoding plasmids, together with a yellow fluorescent protein-(YFP-) encoding plasmid, are introduced into B. napus protoplasts by polyethylene glycol-(PEG-) mediated transformation (Yoo et al., Nature Protocols 2:1565-1572, 2007). Twenty-four hours after treatment, transformation efficiency is measured using a fluorescent microscope to monitor YFP fluorescence in an aliquot of the transformed protoplasts. The remainder of the transformed protoplasts are harvested, and genomic DNA is prepared using a hexadecyltrimethylammonium bromide-(CTAB-) based method. Using genomic DNA prepared from the protoplasts as a template, a fragment encompassing the TALE nuclease recognition site is amplified by PCR. 454 pyro-sequencing is then used to analyze the frequency of NHEJ-induced mutations. Sequencing reads with indel mutations in the spacer region are considered to be derived from imprecise repair of a cleaved TALE nuclease recognition site by NHEJ. Mutagenesis frequency is calculated as the number of sequencing reads with NHEJ mutations out of the total sequencing reads.

### Example 3

### Creating a Donor Matrix for Modifying the B. napus ALS Loci

Recombination donor matrices are prepared to incorporate specific DNA sequence modifications into the ALS loci. These matrices typically have 600 bp of sequence that is homologous to the endogenous ALS loci, with the exception of sequence modifications that introduce the desired mutation(s) to confer herbicide tolerance.

### Example 4

### Regeneration of B. napus Lines with the Intended ALS Gene Edits

B. napus lines are created with mutations in one or more alleles of the ALS gene. Protoplasts are isolated from surface sterilized leaves and transformed with (a) plasmids encoding TALE nucleases targeted to one of the loci, and (b) the corresponding donor matrix. Transformation efficiencies are monitored by delivery of a YFP plasmid, which is visualized using fluorescence microscopy or flow cytometry.

After PEG-mediated transformation, protoplasts are cultured using methods and media described elsewhere (Lian et al., Plant Cell Tiss. Organ Cult. 109:565-572, 2012), with slight modifications. Protoplasts are re-suspended in K8p plating medium at a cell density of 1×10⁵/ml in a small petri dish, and stored at 25° C. in the dark. Three days post transformation, when the majority of the protoplasts have divided at least once, the protoplast culture is resuspended in 1 mL of K8p plating medium with dissolved agarose (Sigma Type VII). Once the colonies reach 1-2 mm in diameter, they are transferred to regeneration medium. These calli are then transferred to fresh media at bi-weekly intervals. Once the calli reach 2-3 mm in diameter they are transferred to shoot-inducing medium. Once roots form, they are transferred to soil and grown to maturity.

### Example 5

### Verification of B. napus Lines with TALE Nuclease-Induced ALS Gene Edits

Once the protoplast-derived B. napus lines are regenerated to plantlets, all alleles of the ALS gene are assessed for mutations. Plants are verified by PCR amplification and subsequent DNA sequencing of the target locus.

### Example 6

### Determining Whether Mutant B. napus Lines have Desired Phenotypes

Determination of the tolerance of mutant B. napus lines to ALS-inhibiting herbicides is assessed via an herbicide response curve. The sensitivity of B. napus callus toward sulfonylureas, imidazolinones, triazolopyrimidine sulfonamides, pyrimidinylsalicylates and sulfonylaminocarbonyl-triazolinones is examined (Sigma-Aldrich). A known amount of protoplast-derived B. napus callus is placed on modified K8p medium (Glimelius et al., Plant Sci. 45:133-141, 1986) and on modified K8p medium containing different concentrations of a single ALS-inhibiting herbicide (between 3 and 500 μg/1). The sensitivity of the protoplast-derived B. napus callus to these ALS-inhibiting herbicides is monitored for 15, weeks with sub-culturing onto fresh medium every 2 weeks (van der Vyver et al., In Vitro Cell Dev Biol—Plant, doi.10.1007/s11627-013-9493-0, 2013).

Determination of whole plant B. napus line tolerance to ALS-inhibiting herbicides is assessed via an herbicide whole plant response curve as described elsewhere (Saari et al., Plant Physiol. 93: 55-61, 1990), with slight modifications. Herbicides are applied to both ALS modified and wild-type B. napus foliage using a laboratory sprayer. The herbicides are dispersed in water containing surfactant, and are used at a range of 0.05 to 75 g ai/ha (grams active ingredient/hectare) to determine the rate of herbicide application required to inhibit plant growth by 50% relative to wild-type B. napus.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A Brassica plant, plant part, or plant cell comprising at least one nucleotide mutation in at least one acetolactate synthase (ALS) allele endogenous to the plant, plant part, or plant cell, such that the plant, plant part, or plant cell has increased tolerance to one or more ALS-inhibiting herbicides as compared to a control Brassica plant, plant part, or plant cell that does not comprise the at least one nucleotide mutation.

2. The plant, plant part, or plant cell of claim 1, wherein the Brassica plant, plant part, or plant cell is a B. napus plant, plant part, or plant cell.

3. The plant, plant part, or plant cell of claim 1, wherein each mutation is a substitution of at least one nucleotide base pair.

4. The plant, plant part, or plant cell of claim 1, wherein the plant, plant part, or plant cell was made using a rare-cutting endonuclease.

5. The plant, plant part, or plant cell of claim 4, wherein the rare-cutting endonuclease is a transcription activator-like effector endonuclease (TALE nuclease), a zinc-finger nuclease, a meganuclease, or a programmable RNA-guided endonuclease.

6. The plant, plant part, or plant cell of claim 4, wherein the rare-cutting endonuclease is a TALE nuclease, a zinc-finger nickase, or a programmable RNA-guided nickase.

7. The plant, plant part, or plant cell of claim 1, wherein each of the at least one ALS allele exhibits substitution of at least one endogenous nucleic acid and does not include any exogenous nucleic acid.

8. The plant, plant part, or plant cell of claim 1, wherein every endogenous ALS allele is modified.

9. The plant, plant part, or plant cell of claim 7, wherein every endogenous ALS allele exhibits substitution of at least one endogenous nucleic acid and does not include any exogenous nucleic acid.

10. The plant, plant part, or plant cell of claim 1, wherein the plant, plant part, or plant cell exhibits increased tolerance to ALS-inhibiting herbicides compared to a control plant, plant part, or plant cell that lacks the mutation.

11. A method for generating a Brassica plant, plant part, or plant cell comprising at least one nucleotide mutation in at least one ALS allele endogenous to the plant, plant part, or plant cell, such that the plant, plant part, or plant cell has increased tolerance to ALS-inhibiting herbicides as compared to a control Brassica plant, plant part, or plant cell that does not comprise the at least one nucleotide mutation, the method comprising:
(a) contacting a Brassica plant, plant part, or plant cell comprising an endogenous ALS gene that can be modified to confer tolerance, with a rare-cutting endonuclease targeted to the endogenous ALS gene;
(b) contacting the Brassica plant, plant part, or plant cell with a donor matrix comprising a sequence homologous to the endogenous ALS gene, with the exception of the at least one nucleotide mutation; and
(c) growing the selected plant part or plant cell into a Brassica plant, wherein the Brassica plant has increased tolerance to ALS-inhibiting herbicides as compared to the control Brassica plant;
to produce a Brassica plant, plant part, or plant cell having tolerance to ALS-inhibiting herbicides, in which homologous recombination of the donor matrix has conferred the tolerance.

12. The method of claim 11, wherein the Brassica plant cells are protoplasts.

13. The method of claim 12, further comprising culturing the protoplasts to generate plant lines.

14. The method of claim 12, comprising isolating genomic DNA comprising at least a portion of the ALS loci from the protoplasts.

15. The method of claim 11, wherein the rare-cutting endonuclease is encoded by a nucleic acid.

16. The method of claim 15, wherein the nucleic acid is an mRNA.

17. The method of claim 15, wherein the nucleic acid is contained within a vector.

18. The method of claim 17, wherein the vector is a viral vector.

19. The method of claim 11, wherein the rare-cutting endonuclease is a protein.

20. The method of claim 11, wherein the rare-cutting endonuclease is a TALE nuclease, a zinc-finger nuclease, a meganuclease, or a programmable RNA-guided endonuclease.

21. The method of claim 11, wherein the rare-cutting endonuclease is a transcription activator-like effector nickase, a zinc-finger nickase, or a programmable RNA-guided nickase.

22. The method of claim 11, wherein the donor matrix comprises a single-stranded nucleic acid.

23. The method of claim 22, wherein the single-stranded nucleic acid is a ribonucleic acid.

24. The method of claim 11, wherein the donor matrix is contained within a vector.

25. The method of claim 24, wherein the vector is a viral vector.

26. The method of claim 11, wherein the Brassica plant cells are B. napus plant cells.
